(19) United States
(12) Patent Application Publication
(10) Pub. No.: US 2024/0058261 A1
(43) Pub. Date: Feb. 22, 2024

(54) ANTI-AGING COMPOSITION COMPRISING FERMENTED PROPOLIS

(71) Applicant: LG HOUSEHOLD & HEALTH CARE LTD., Seoul (KR)

(72) Inventors: Youn Jung LEE, Seoul (KR); Tae Ho JI, Seoul (KR); Woon Ki BAEK, Seoul (KR)

(73) Assignee: LG HOUSEHOLD & HEALTH CARE LTD., Seoul (KR)

(21) Appl. No.: 18/258,282

(22) PCT Filed: Dec. 14, 2021
(86) PCT No.: PCT/KR2021/018946

§ 371 Date: Jun. 19, 2023

(30) Foreign Application Priority Data
Dec. 18, 2020 (KR) 10-2020-0178888
Dec. 9, 2021 (KR) 10-2021-0175504

(51) Int. Cl. A61K 8/98; A61Q 19/00

(52) U.S. Cl. CPC A61K 8/988; A61Q 19/00; A61K 2800/85

(57) ABSTRACT

A composition including a fermented propolis extract as an active ingredient and a method of preparing same. The composition including a fermented propolis extract as an active ingredient, according to the present invention, can be used for various applications such as improvement in skin wrinkles or skin elasticity, skin whitening, skin moisturizing, and skin antioxidant or anti-inflammatory applications. In addition, the composition can exhibit stronger effects due to the content of artepillin C that is increased by a preparation method according to the present invention.

### TECHNICAL FIELD

The present invention relates to a composition including a fermented propolis extract as an active ingredient and a preparation method thereof.

### BACKGROUND ART

Propolis, which is also called Russian penicillin or natural penicillin, is a natural waxy mixture made after bees collect resins, such as growing point-protecting substances, sap, and the like, from trees and plants and the resins are mixed with saliva that the bees secrete. Propolis is composed of 300 or more substances such as vegetable resins, waxes, essential oils, pollen, flavonoids, other organic substances, minerals, and the like.

The main effects of propolis include anti-inflammation, antioxidation, anti-cancer, immune enhancement, and the like. Recently, various pharmacological actions resulting from the antioxidant action of flavonoids and terpenoids, which are components included in propolis, have been studied and attracted considerable interest. Particularly, research has shown that the therapeutic effect on skin, mucous membranes, and oral infections results from various substances isolated and identified from propolis, and phenylpropane derivatives such as at least 150 terpenes, caffeic acid esters, flavonoids, and amino acids are involved. As studies on the use of propolis for the relief of inflammatory diseases are gradually increasing, various products using propolis are also being developed.

Types of propolis differ in active ingredients, color, taste, and the like depending on the type and distribution of plants that inhabit each region, and this is because the components differ depending on the type of tree sap or resins collected by working bees. The color of propolis varies, and there are differences in active ingredients according to a difference in color. For example, green propolis, red propolis, brown propolis, and the like are known.

Meanwhile, microorganisms have been usefully used in conventional food. Recently, the use of microorganisms in pharmaceutical manufacturing and cosmetic industries is also increasing.

Lactic acid bacteria are microorganisms that produce lactic acid using carbohydrates anaerobically. The lactic acid bacteria have been used as raw materials for cosmetics. For example, fermented liquids obtained by culturing lactic acid bacteria such as Streptococcus sp., Pediococcus sp., Leuconostoc sp., Lactobacillus sp., Sporolactobacillus sp., Bifidobacterium sp., and the like have been conventionally reported to have a whitening or moisturizing effect, but there is no literature which discloses that, when propolis is fermented with the above strains of lactic acid bacteria, skin wrinkling, skin elasticity, skin whitening, skin antioxidant, skin moisturizing, and skin anti-inflammatory effects are enhanced.

Meanwhile, among the types of propolis, green propolis is known to be highly effective for antioxidation, anti-inflammation, and the like due to containing artepillin C unlike other types of propolis.

Since a trace amount of artepillin C is extracted when extracted by a general extraction method, there is a need for a method of preparing an extract while increasing an artepillin C content.

### RELATED ART DOCUMENTS

### Patent Documents

1: KR 10-2298533

### DISCLOSURE

### Technical Problem

The present invention is directed to providing a composition for skin wrinkle improvement, elasticity improvement, whitening, antioxidation, or anti-inflammation, which includes a fermented propolis extract as an active ingredient.

The present invention is also directed to providing a method of preparing a fermented propolis extract having an increased artepillin C content and fermented propolis prepared by the method.

### Technical Solution

As a result of research on a method of increasing an artepillin C content in propolis, that is, in an extract when the extract is prepared, to achieve the above objectives of the present invention, the inventors of the present invention have found that, when extraction is made by cold extraction, ultrasonic extraction, inoculation of fermentation strains, and solvent extraction, a fermented propolis extract having an increased artepillin C content can be obtained, and thereby completed the present invention.

Therefore, one aspect of the present invention provides a composition including a fermented propolis extract as an active ingredient. The composition according to the present invention may have an increased artepillin C content.

In the present invention, artepillin C may have a molecular formula of C₁₉H₂₄O₃ and a molecular weight of 300.40 and may be represented by the following Chemical Formula 1.

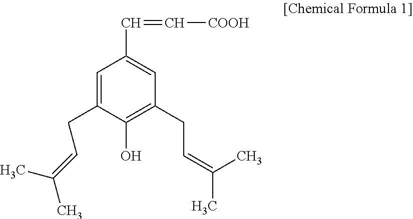 [SMILES: CC(C)=CCc1cc(C=CC(=O)O)cc(CC=C(C)C)c1O]

In the present invention, there is no limitation on a type of propolis, but the propolis may be one or more selected from the group consisting of green propolis, red propolis, and brown propolis and is preferably green propolis.

Meanwhile, artepillin C, which is known to be contained in a large amount in green propolis, is a component conventionally known to be effective for whitening, antioxidation (“Artepillin C and phenolic compounds responsible for antimicrobial and antioxidant activity of green propolis and Baccharis dracunculifolia DC,” M. C. Marcucci et al., Journal of Applied Microbiology 122, 911-920), anti-inflammation (“Anti-inflammatory effects of a bioavailable compound, Artepillin C, in Brazilian propolis,” Walter A. Bretz et al., European Journal of Pharmacology 587 (2008) 296-301), moisturizing, and the like.

Another aspect of the present invention provides a method of preparing a fermented propolis extract having an increased artepillin C content.

According to an embodiment, an amount of artepillin C contained in a fermented propolis extract prepared by the method according to the present invention is 35 ppm, and it was confirmed that the artepillin C content is increased about 5.8 times relative to 6 ppm which is an amount of artepillin C contained in a propolis extract obtained by a general extraction method.

In the case of the composition of the present invention, the amount of artepillin C contained in propolis may be increased by cold extraction, ultrasonic extraction, inoculation of fermentation strains, and solvent extraction, and accordingly, better effects of improving skin wrinkles or skin elasticity, skin whitening, skin moisturizing, skin antioxidation, and/or anti-inflammation can be provided. In this regard, the present invention provides a composition for improving skin wrinkles or skin elasticity, skin whitening, skin moisturizing, skin antioxidation, and/or anti-inflammation. The composition may be used as a cosmetic composition, a pharmaceutical composition, a composition for external application, a quasi-drug composition, or a food composition, and each composition may be used in varying categories depending on the purpose of use, the content of an active ingredient, and the like.

In the present specification, a “wrinkle improvement effect” refers to an effect of preventing, inhibiting, or suppressing the formation of wrinkles on skin or alleviating wrinkles that have already been formed.

In the present specification, an “elasticity improvement effect” refers to an effect of maintaining or improving skin elasticity.

In the present specification, a “lifting improvement effect” refers to an effect of physically pulling up the skin tissue due to skin elasticity and wrinkle improvement effects.

According to an embodiment of the present invention, it was confirmed that, when a human fibroblast medium is treated with a propolis extract and a fermented propolis extract obtained by fermenting a propolis extract with a fermentation strain, a group treated with a propolis extract before fermentation does not exhibit a cell activation effect, whereas a group treated with a fermented propolis extract after fermentation exhibits a cell activation effect.

In other embodiments of the present invention, the effects of a propolis extract on improvement in around-eye wrinkles, under-eye wrinkles, nasolabial folds, and neck wrinkles (FIGS. 4 to 7), lifting improvement effects in the butterfly zone of the left and right cheeks, around the mouth, around the eyes, in the nasolabial area, in the double chin area, and in the chin area (FIGS. 8 to 14), skin density and skin elasticity improvement effects (FIGS. 15 and 16), an improvement in melasma area (FIG. 17), an skin radiance increase effect (FIG. 18), and a skin moisturizing improvement effect (FIGS. 19 and 20) were confirmed.

The above-described effects of the present invention may be exhibited due to an increased artepillin C content in a fermented propolis extract prepared by the method of the present invention.

The composition of the present invention may be a composition for improving skin wrinkles or skin elasticity, which includes a fermented propolis extract as an active ingredient.

In the present specification, a “whitening effect” refers to not only an effect of brightening the skin tone but also an effect of relieving or improving melasma, freckles, and hyperpigmentation, which are caused by ultraviolet rays, hormones, or heredity.

In the present specification, a “radiance effect” refers to an effect of maintaining a moist skin condition due to skin moisturizing and a clear skin tone due to skin whitening.

The composition of the present invention may be a composition for skin whitening, which includes a fermented propolis extract as an active ingredient.

In the present invention, “moisturizing” refers to an action of supplying moisture to dry, rough and flaky skin caused by dry air and stimulation, and blocking evaporation of moisture to maintain the flexibility of the skin and induce uniform exfoliation of dead skin cells, thereby maintaining a smooth surface.

The moisturizing may be, for example, for one or more selected from the group consisting of atopic dermatitis, psoriasis, xeroderma, eczema, and xeroderma pigmentosum, but the present invention is not limited thereto.

Therefore, the composition of the present invention may be a composition for skin moisturizing, which includes a fermented propolis extract as an active ingredient.

In the present invention, an “antioxidant effect” refers to an effect of inhibiting cell oxidation caused by highly reactive free radicals or reactive oxygen species (ROS) under oxidative stress caused by intracellular metabolism or ultraviolet rays, and includes removing free radicals or reactive oxygen species to reduce damage to cells.

Therefore, the composition of the present invention may be a composition for skin antioxidation, which includes a fermented propolis extract as an active ingredient.

In the present invention, an “anti-inflammatory effect” refers to an effect of inhibiting inflammation, and inflammation is one of the defense responses of biological tissues to certain stimuli and refers to a complex lesion in which tissue deterioration, circulatory disturbance and exudation, and tissue proliferation occur. More specifically, inflammation is part of innate immunity, and as in other animals, innate immunity in humans recognizes cell surface patterns that are specific to pathogens. Phagocytes recognize cells with such surfaces as a non-self and attack the pathogen. When pathogens break through the physical barrier of the body and enter the body, inflammatory responses occur. The inflammatory response is a non-specific defense action that creates a hostile environment for microorganisms invading a wound. In the inflammatory response, when a wound is formed or an external infectious agent enters the body, white blood cells in charge of an initial stage of the immune response flock to express cytokines. Therefore, the expression level of intracellular cytokines is an indicator for activation of the inflammatory response. Examples of skin diseases related to inflammation include atopic dermatitis, psoriasis, erythematous diseases triggered by radiation, chemicals, burns, and the like, acid burns, bullous dermatosis, lichenoid diseases, itching caused by allergies, seborrheic eczema, rosacea, pemphigus vulgaris, exudative erythema multiforme, erythema nodosum, balanitis, vulvitis, inflammatory hair loss such as alopecia areata, cutaneous T-cell lymphoma, and the like, but the present invention is not limited thereto.

Therefore, the composition of the present invention may be a composition for anti-inflammation, which includes a fermented propolis extract as an active ingredient.

In the present invention, a method of obtaining propolis is not particularly limited, and propolis may be obtained by extraction from a natural substance and/or purification, may be chemically synthesized by a method known in the art, or may be a commercially available substance.

According to an embodiment, the propolis of the present invention may be derived from a substance known as “propolis” or “wax,” but the present invention is not limited thereto.

In the cosmetic composition, pharmaceutical composition, composition for external application, quasi-drug composition, or food composition according to the present invention, the fermented propolis extract may be included in an amount of 0.01 to 15 wt %, preferably, 0.1 to 10 wt % relative to the total weight of the cosmetic composition, pharmaceutical composition, composition for external application, quasi-drug composition, or food composition.

In the present invention, the term “extract” includes an extract itself and extracts in varying formulations that may be formed using a liquid extract, such as a liquid extract obtained by extraction of propolis, a diluted or concentrated solution of the liquid extract, a dry material obtained by drying the liquid extract, a crude or purified product of the liquid extract, or a mixture thereof.

Meanwhile, the method of preparing a fermented propolis extract having an increased artepillin C content includes: a first step of subjecting propolis wax to cold extraction to prepare a propolis extract; a second step of subjecting the propolis extract extracted in the first step to ultrasonic extraction; a third step of fermenting the extract obtained in the second step with a fermentation strain; and a fourth step of subjecting the extract fermented in the third step to solvent extraction with 1,3-butylene glycol.

A cold extraction temperature may be 5 to 40° C. or 5 to 25° C., preferably, 10 to 20° C., and a cold extraction time may be 200 to 400 hours or 230 to 370 hours, preferably, 250 to 350 hours.

In addition, the cold extraction may be solvent extraction with a C1 to C5 alcohol, and the alcohol may be, for example, ethanol, propyl alcohol, butyl alcohol, or the like, preferably, ethanol.

When the solvent used in the cold extraction is not a C1 to C5 alcohol, the active ingredient in the fermented propolis extract of the present invention may not be extracted in an effective amount.

In the ultrasonic extraction, a wavelength may be 10 kHz to 40 kHz, a temperature may be 10° C. to 40° C., and a treatment time may be 30 minutes to 2 hours.

When the wavelength, temperature, and treatment time conditions of the ultrasonic extraction are not satisfied, the active ingredient in the fermented propolis extract of the present invention may not be extracted in an effective amount.

In the present invention, the propolis extract may be fermented with a fermentation strain after being subjected to ultrasonic extraction. There is no limitation on the type of fermentation strain, but the fermentation strain may be one or more selected from the group consisting of Lactobacillus acidophilus, Lactobacillus casei, Lactobacillus bulgaricus, Lactobacillus rhamnosus, Lactobacillus plantarum, Lactobacillus reuteri, Lactobacillus confusus, Lactobacillus fermentum, Lactobacillus brevis, and Thermus thermophilus. A fermentation time may be 24 to 120 hours (1 to 5 days), preferably, 48 to 96 hours (2 to 4 days).

Extraction may be performed by adding 1,3-butylene glycol in an amount of 20 to 40 wt % or 23 to 37 wt %, preferably, 25 to 35 wt % based on the total weight of the extract obtained after the fermentation, and then a fermented propolis extract having an increased artepillin C content may be finally obtained.

When 1,3-butylene glycol does not satisfy the above-described amount (wt %) or a solvent other than 1,3-butylene glycol is used, the dissolution stability of an extract is degraded, and thus precipitation may occur. Also, preservative efficacy is degraded, and thus microorganisms may proliferate.

In an embodiment, a fermented propolis extract obtained by the above preparation method was confirmed to have an artepillin C content about 5.8 times higher than a conventionally known propolis extract.

Therefore, the present invention provides a composition which includes a fermented propolis extract as an active ingredient and has an increased artepillin C content.

Meanwhile, the composition according to the present invention may be prepared in the form of any one selected from the group consisting of a solution, an ointment for external use, a cream, a foam, a nutritional skin toner, a skin softening toner, a pack, softening water, a lotion, a makeup base, an essence, soap, a cleanser, a bath bomb, a sunscreen cream, a sunscreen oil, a suspension, an emulsion, a paste, a gel, a lotion, a powder, soap, a surfactant-containing cleansing, an oil, a powder foundation, an emulsion foundation, a wax foundation, a patch, and a spray, preferably, in the form of a toner, an essence, a lotion, a cream, a pack, a gel, a powder, a foundation, or a cleanser, but the present invention is not limited thereto.

The cosmetic composition according to the present invention may further include one or more cosmetically acceptable carriers that are mixed in general skin cosmetics, and typical ingredients, for example, oil, water, a surfactant, a moisturizing agent, a lower alcohol, a thickening agent, a chelating agent, a pigment, a preservative, a fragrance, and the like, may be appropriately mixed, but the present invention is not limited thereto.

When the formulation of the present invention is a powder or spray, lactose, talc, silica, aluminum hydroxide, calcium silicate, polyamide powder, or a mixture thereof may be used as a carrier ingredient. Particularly, when the formulation is a spray, a propellant such as chlorofluorohydrocarbon, propane/butane, or dimethyl ether may be further included.

When the formulation of the present invention is a solution or emulsion, a solvent, a solubilizing agent, or an emulsifying agent may be used as a carrier ingredient. For example, water, ethanol, isopropanol, ethyl carbonate, ethyl acetate, benzyl benzoate, propylene glycol, 1,3-butyl glycol oil, particularly, cottonseed oil, peanut oil, corn germ oil, olive oil, castor oil, and sesame oil, aliphatic esters of glycerol, polyethylene glycol, or fatty acid esters of sorbitan may be used.

When the formulation of the present invention is a suspension, a liquid diluent such as water, ethanol, or propylene glycol, a suspending agent such as ethoxylated isostearyl alcohol, polyoxyethylene sorbitol ester, or polyoxyethylene sorbitan ester, microcrystalline cellulose, aluminum metahydroxide, bentonite, agar, tragacanth, or the like may be used as a carrier ingredient.

When the formulation of the present invention is soap, an alkali metal salt of fatty acids, a fatty acid hemiester salt, a fatty acid protein hydrolysate, an isethionate, a lanolin derivative, an aliphatic alcohol, a vegetable oil, glycerol, sugar, or the like may be used as a carrier ingredient.

In addition, the present invention provides a method of improving skin wrinkles or skin elasticity, skin whitening, skin moisturizing, skin antioxidation, or preventing, improving, or treating inflammation, which includes applying a composition including a fermented propolis extract as an active ingredient to a subject's skin.

Still another aspect of the present invention provides a method of improving skin wrinkles or skin elasticity, which includes administering or applying a composition including a fermented propolis extract to a subject or subject's skin and improving skin wrinkles or skin elasticity in the subject or subject's skin.

Yet another aspect of the present invention provides a skin whitening method, which includes administering or applying a composition including a fermented propolis extract to a subject or subject's skin and improving skin whitening in the subject or subject's skin.

Yet another aspect of the present invention provides a skin antioxidation method, which includes administering or applying a composition including a fermented propolis extract to a subject or subject's skin and inhibiting or reducing oxidation in the subject or subject's skin.

Yet another aspect of the present invention provides a method of preventing, improving, or treating skin inflammation, which includes administering or applying a composition including a fermented propolis extract to a subject or subject's skin and inhibiting or reducing the occurrence of inflammation in the subject or subject's skin.

Yet another aspect of the present invention provides a skin moisturizing method, which includes administering or applying a composition including a fermented propolis extract to a subject or subject's skin and providing moisturizing to the subject or subject's skin.

The subject includes mammals including humans, livestock, rats, and the like without limitation.

Yet another aspect of the present invention provides a pharmaceutical composition for improving skin wrinkles or skin elasticity, skin whitening, skin moisturizing, skin antioxidation, or anti-inflammation, which includes a fermented propolis extract as an active ingredient.

In the present invention, the term “pharmaceutical composition” may be used as a concept that encompasses “quasi-drugs” or “drugs.”

The composition of the present invention may be in various oral or parenteral formulations, and is preferably in a parenteral formulation. When the composition is formulated, the composition may be prepared using a commonly used diluent or excipient such as a filler, an extending agent, a binding agent, a wetting agent, a disintegrating agent, a surfactant, or the like. A solid preparation for oral administration includes a tablet, a pill, a powder, granules, a capsule, and the like. Such a solid preparation is prepared by mixing one or more compounds with at least one excipient, for example, starch, calcium carbonate, sucrose or lactose, gelatin, or the like. Also, in addition to the simple excipient, a lubricant such as magnesium stearate, talc, or the like is used. A liquid preparation for oral administration includes a suspending agent, a liquid for internal use, an emulsion, a syrup, and the like. In this case, the liquid preparation for oral administration may include various excipients such as a wetting agent, a sweetening agent, a fragrance, a preservative, and the like in addition to generally used simple diluents such as water and liquid paraffin. A preparation for parenteral administration includes a sterile aqueous solution, a non-aqueous solvent, a suspending agent, and an emulsion. Propylene glycol, polyethylene glycol, a vegetable oil (such as olive oil), an injectable ester (such as ethyl oleate), and the like may be used as a non-aqueous solvent and a suspending agent.

Yet another aspect of the present invention provides a composition for external application for improving skin wrinkles or skin elasticity, skin whitening, skin moisturizing, skin antioxidation, or anti-inflammation, which includes a fermented propolis extract as an active ingredient.

When the composition including a fermented propolis extract as an active ingredient is used as an external preparation for skin, an adjuvant typically used in the field of dermatology, such as a lipid material, an organic solvent, a solubilizing agent, a concentrate, a gelating agent, an emollient, an antioxidant, a suspending agent, a stabilizer, a foaming agent, a fragrance, a surfactant, water, an ionic emulsifier, a non-ionic emulsifier, a filler, a sequestering agent, a chelating agent, a preservative, a vitamin, a blocking agent, a wetting agent, an essential oil, a dye, a pigment, a hydrophilic active agent, a lipophilic active agent, a lipid vesicle, and any other ingredients typically used in an external preparation for skin may be further contained. Also, the ingredients may be introduced in amounts generally used in the field of dermatology.

When the compound of Chemical Formula 1 is provided as a composition for external application, it may be in a formulation such as an ointment, a patch, a gel, a cream, and a spray, but the present invention is not limited thereto.

Particularly preferably, the composition for external application of the present invention may be used as a parenteral preparation. For example, the composition for external application may be prepared by a typical method of preparing a composition for external skin application, in which a suitable pharmaceutically acceptable base such as Vaseline, stearyl alcohol, and the like; a suitable pharmaceutically acceptable surfactant such as polysorbate, sorbitan sesquioleate, and the like; a suitable pharmaceutically acceptable moisturizing agent such as glycerin and the like; a suitable pharmaceutically acceptable solvent; and a fragrance, a coloring agent, a stabilizing agent, a thickening agent, and the like are homogeneously mixed.

Yet another aspect of the present invention provides a quasi-drug composition for improving skin wrinkles or skin elasticity, skin whitening, skin moisturizing, skin antioxidation, or anti-inflammation, which includes a fermented propolis extract as an active ingredient.

When the composition including a fermented propolis extract as an active ingredient is provided as a quasi-drug composition, the quasi-drug composition includes one or more compounds selected from the group consisting of hydroxycinnamic acid, isoamyl acetate, and betaine or a pharmaceutically acceptable salt thereof as an active ingredient. In addition, as necessary, the quasi-drug composition may further include a pharmaceutically acceptable carrier, excipient, or diluent. The pharmaceutically acceptable carrier, excipient, or diluent is not limited as long as it does not hinder the effects of the present invention, and for example, a filler, an extending agent, a binding agent, a wetting agent, a disintegrating agent, a surfactant, a lubricant, a sweetener, a fragrance, a preservative, and the like may be included.

As the quasi-drug composition of the present invention, a disinfectant cleanser, a shower foam, an ointment, a wet tissue, a coating agent, and the like may be exemplified. Preferably, the quasi-drug composition may be prepared in a semi-solid preparation such as an ointment for external use or a lotion, but the present invention is not limited thereto. The preparation method, capacity, method of use, and components of the quasi-drug composition may be appropriately selected from conventional technique known in the art.

Yet another aspect of the present invention provides a food composition for improving skin wrinkles or skin elasticity, skin whitening, skin moisturizing, skin antioxidation, or anti-inflammation, which includes a fermented propolis extract as an active ingredient.

When the composition including a fermented propolis extract as an active ingredient is provided as a food composition, the composition may include a sitologically acceptable food supplement additive in addition to an active ingredient.

The food supplement additive refers to a component that may be added to food, and an additive added to prepare a health functional food in varying formulations may be appropriately selected by those skilled in the art. Examples of the food supplement additive include various nutrients, vitamins, minerals (electrolytes), flavoring agents such as artificial flavoring agents, natural flavoring agents and the like, coloring agents and fillers, pectic acid and salts thereof, alginic acid and salts thereof, organic acids, protective colloidal thickening agents, pH controlling agents, stabilizers, preservatives, glycerin, alcohols, carbonizing agents as used in carbonated beverages, and the like, but the type thereof is not limited thereto.

In addition, the food composition may include a health functional food. In the present invention, the health functional food refers to a food group with added value so that the function of the corresponding food acts and is expressed for a specific purpose using physical, biochemical, and bioengineering techniques or a food designed and processed to sufficiently express the body's regulatory functions related to biological defense rhythm control, disease prevention, and recovery. Meanwhile, a health food refers to a food that has an active health maintenance or enhancement effect compared to general food, and a health supplement food refers to a food for the purpose of supplementing health. In some cases, a functional food, health food, and health supplement food are interchangeably used.

Specifically, the health functional food is a food prepared by adding the composition of the present invention to food materials such as beverages, teas, spices, chewing gum, confectioneries, or the like or prepared in the form of a capsule, powder, suspension, or the like. When ingested, the food has a specific health effect, and since a food is used as a raw material unlike general drugs, there is no side effect that may occur when the drug is taken for a long time.

The food may include a sitologically acceptable food supplement additive and may further include an appropriate carrier, excipient, and diluent, which are typically used in production of health functional food.

The composition may include an additional ingredient that is typically used in a food composition to improve smell, taste, appearance, and the like. For example, the composition may include vitamins A, C, D, E, B1, B2, B6, B12, niacin, biotin, folate, pantothenic acid, or the like. Also, the composition may include a mineral such as zinc (Zn), iron (Fe), calcium (Ca), chromium (Cr), magnesium (Mg), manganese (Mn), copper (Cu), chromium (Cr), or the like. Also, the composition may include an amino acid such as lysine, tryptophan, cysteine, valine, or the like. There is no limitation on the type of health functional food of the present invention, and it may include all food in a conventional sense and may be used interchangeably with terms known in the art, such as functional food.

In addition, the health functional food of the present invention may be prepared by mixing other suitable auxiliary ingredients and known additives that may be included in food according to the selection of those skilled in the art. Examples of food include meat, sausages, bread, chocolate, candies, snacks, cookies, pizza, ramen, other noodles, chewing gum, dairy products including ice cream, various types of soup, beverages, tea, health drinks, alcoholic drinks, vitamin complexes and the like, and juice, tea, jelly, and juice prepared using the extract according to the present invention as a main ingredient may also be included. Also, food used as feed for animals may be included.

The composition of the present invention may further include an ingredient for improving skin wrinkles or skin elasticity, skin whitening, skin moisturizing, skin antioxidation, or anti-inflammation, which is known in the art. The additional ingredient may be included in an amount of 0.0001 wt % to 10 wt % with respect to the total weight of the composition, and the content range may be adjusted according to requirements such as skin safety, ease of formulation, and the like.

The above-described ingredients included in the cosmetic composition according to the present invention may be included in the cosmetic composition of the present invention within a range not exceeding the maximum amount specified in standards related to “Cosmetic Use and Permission” prescribed by each government.

In the present invention, an “effective amount” refers to an amount of an extract, which is sufficient for exhibiting an effect of improving skin wrinkles or skin elasticity, skin whitening, skin moisturizing, skin antioxidation, or anti-inflammation.

Numerical values described herein above should be construed as including equivalent ranges unless otherwise specified.

### Advantageous Effects

The composition including a fermented propolis extract as an active ingredient according to the present invention can be used for various applications such as skin wrinkle or skin elasticity improvement, skin whitening, skin moisturizing, skin antioxidation, and anti-inflammation, and since an artepillin C content of the composition is increased by the preparation method according to the present invention, more excellent effects are exhibited, and thus an anti-aging effect can be exhibited.

### DESCRIPTION OF DRAWINGS

FIG. 1 shows that a propolis extract before fermentation according to a preparation method of the present invention has no effect on cell activity.

FIG. 2 shows that a propolis extract after fermentation according to a preparation method of the present invention increases cell activity compared to a negative control.

FIG. 3 shows the antioxidant effect of a fermented propolis extract of the present invention. It can be seen that the fermented propolis extract of the present invention scavenges 50% of radicals at a concentration (IC50) of 2.91±0.12%.

FIG. 4 shows the around-eye wrinkle improvement effect of a fermented propolis extract of the present invention as a wrinkle improvement effect.

FIG. 5 shows the under-eye wrinkle improvement effect of a fermented propolis extract of the present invention as a wrinkle improvement effect.

FIG. 6 shows the nasolabial fold improvement effect of a fermented propolis extract of the present invention as a wrinkle improvement effect.

FIG. 7 shows the neck wrinkle improvement effect of a fermented propolis extract of the present invention as a wrinkle improvement effect.

FIG. 8 shows a lifting improvement effect of a fermented propolis extract of the present invention in the butterfly zone (left cheek).

FIG. 9 shows the lifting improvement effect of a fermented propolis extract of the present invention in the butterfly zone (right cheek).

FIG. 10 shows the lifting improvement effect of a fermented propolis extract of the present invention around the mouth.

FIG. 11 shows the lifting improvement effect of a fermented propolis extract of the present invention around the eyes.

FIG. 12 shows the lifting improvement effect of a fermented propolis extract of the present invention in the nasolabial area.

FIG. 13 shows the lifting improvement effect of a fermented propolis extract of the present invention in the double chin area.

FIG. 14 shows the lifting improvement effect of a fermented propolis extract of the present invention in the chin area.

FIG. 15 shows the skin density improvement effect of a fermented propolis extract of the present invention.

FIG. 16 shows the skin elasticity improvement effect of a fermented propolis extract of the present invention.

FIG. 17 shows the melasma area improvement effect of a fermented propolis extract of the present invention.

FIG. 18 shows the skin radiance improvement effect of a fermented propolis extract of the present invention.

FIG. 19 shows the skin moisturizing improvement effect of a fermented propolis extract of the present invention.

FIG. 20 shows the skin deep moisturizing improvement effect of a fermented propolis extract of the present invention.

FIG. 21 shows an example of analysis of the lifting angle of a fermented propolis extract of the present invention in the butterfly zone, around the mouth, and around the eyes.

FIG. 22 shows an example of analysis of the chin lifting angle of a fermented propolis extract of the present invention.

FIG. 23 shows that a fermented propolis extract of the present invention exhibits a higher antioxidant effect than a general propolis extract and maintains antioxidant activity well even at high temperature.

### MODES OF THE INVENTION

Hereinafter, the present invention will be described in detail with reference to the following examples. However, it should be understood that the following examples are given for the purpose of illustration only and are not intended to limit the scope of the present invention.

### Preparation Example 1: Preparation of Fermented Green Propolis Extract

In this preparation example, an extract is prepared using green propolis which is one type of propolis, and a preparation method thereof is as follows.
1. Primary extraction: Green propolis wax was subjected to cold extraction with ethanol as a solvent at 10° C. for 300 hours.
2. Secondary extraction: The extract obtained in the primary extraction was subjected to ultrasonic extraction for an hour by applying ultrasonic waves at room temperature and 20 kHz.
3. The resulting product obtained in the secondary extraction was filtered, concentrated (evaporated), and sterilized (autoclaved).
4. The obtained green propolis extract was inoculated with lactic acid bacteria (Lactobacillus acidophilus) in a culture medium, and fermented (cultured) for 72 hours.
5. After sterilization and filtration, 1,3-butylene glycol was added so that an amount thereof became 30%, and filtration was performed once again to prepare a fermented green propolis extract.

### Comparative Example 1: Preparation of Green Propolis Extract

1. Green propolis wax was thermally extracted by heating in a 50:50 solution of 1,3-butylene glycol and distilled water as a solvent at 60° C. and stirring for an hour.
2. The resulting product was primarily filtered and allowed to cool for 3 days.
3. After 3 days, secondary filtration was performed.
4. For preservative purposes, 1,2-haxandiol was added so that an amount thereof became 2 wt %, and filtration was performed once again to prepare a green propolis extract.

### Preparation Example 2: Preparation of Composition Including Fermented Green Propolis Extract

Compositions including the fermented green propolis extract prepared by the above process were prepared as shown in Table 1 (Examples) and Table 2 (Comparative Example).

TABLE 1

| | Examples | | | |
|---|---|---|---|---|
| Classification (wt %) | 1 | 2 | 3 | 4 |
| Fermented green propolis extract | 1 | 4 | 9 | 15 |
| Butylene glycol | 1 | 1 | 1 | 1 |
| Glycerin | 5 | 5 | 5 | 5 |
| Carbomer | 0.2 | 0.2 | 0.2 | 0.2 |
| Arginine | 0.2 | 0.2 | 0.2 | 0.2 |
| Fragrance | 0.1 | 0.1 | 0.1 | 0.1 |
| Purified water | re- | re- | re- | re- |
| | mainder | mainder | mainder | mainder |

TABLE 2

| Classification (wt %) | Example 3 | Comparative Example 2 |
|---|---|---|
| Green propolis extract | — | 9 |
| Fermented green propolis extract | 9 | — |
| Butylene glycol | 1 | 1 |
| Glycerin | 5 | 5 |
| Carbomer | 0.2 | 0.2 |
| Arginine | 0.2 | 0.2 |
| Fragrance | 0.1 | 0.1 |
| Purified water | remainder | remainder |

### Experimental Example 1: Analysis and Comparison of Artepillin C Content

The content of artepillin C (powerful antioxidant ingredient), which is a standard ingredient of green propolis, was analyzed using liquid chromatography.

TABLE 3

| | Artepillin C (ppm) |
|---|---|
| Preparation Example 1 | 35 |
| Comparative Example 1 | 6 |

### Preparation Example 1: Fermented Green Propolis Extract Prepared by the Method of the Present Invention

### Comparative Example 1: General Green Propolis Extract

As a result of analyzing the artepillin C content, an approximate 5.8 times larger amount of artepillin C was detected in the fermented green propolis extract prepared by the method of the present invention.

### Experimental Example 2: Comparison of Cell Activity Before and After Fermentation of Green Propolis Extract (In Vitro)

The cell activation effect of the sample in a human fibroblast culture was evaluated.

The sample was diluted and treated for 48 hours according to each concentration, and then cell activity was confirmed using a CCK-8 kit.

The cell activity of the sample was determined by calculating an increase rate relative to a negative control (0% serum-containing DMEM).

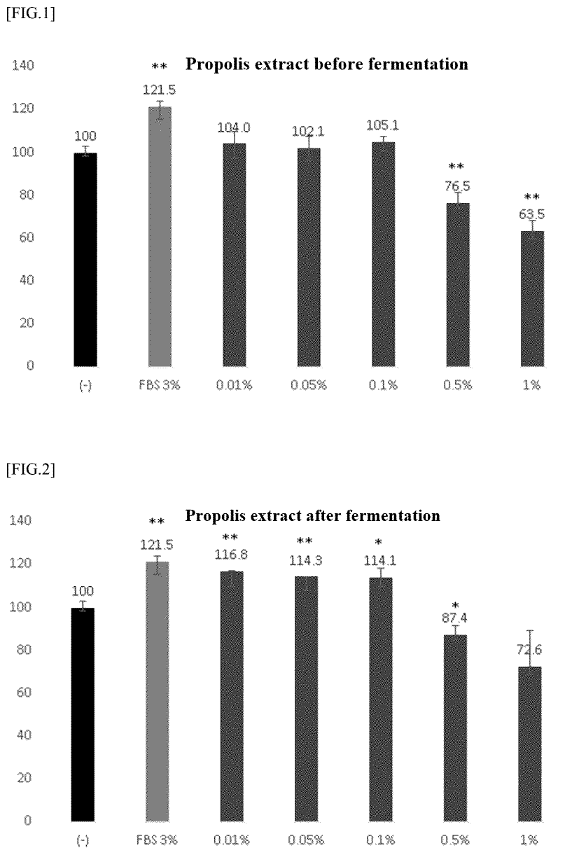

As a result, the green propolis extract before fermentation (Comparative Example 1) had no cell activation effect, and when the green propolis extract after fermentation was treated at 0.01 wt % relative to the total weight of the composition in the medium, cell activity was increased 14% compared to a negative control (FIGS. 1 and 2).

### Experimental Example 3: Antioxidant Effect of Fermented Green Propolis Extract (In Vitro)

In order to confirm the antioxidant effect of the fermented green propolis extract prepared in Preparation Example 1, the antioxidant effect was evaluated by a radical scavenging activity test (DPPH test) using the fact that, when radicals present in 2,2-diphenyl-1-picrylhydrazyl (DPPH) are scavenged, the color of DPPH changes from purple to yellow.

DPPH was purchased from Sigma-Aldrich Inc. (USA).

First, the fermented green propolis extract of Preparation Example 1 was dissolved in DMSO, and then a radical scavenging activity test was performed. A 0.15 mM DPPH solution and the fermented green propolis extract were mixed in equal amounts (100 μl) and allowed to react at room temperature for 30 minutes, and a change in DPPH color was determined by measuring absorbance at 540 nm (A₅₄₀). An IC₅₀ value was calculated using the measured A540 value, and radical scavenging activity was comparatively evaluated.

The IC₅₀ refers to the concentration of the fermented green propolis extract or vitamin C required to scavenge 50% of radicals.

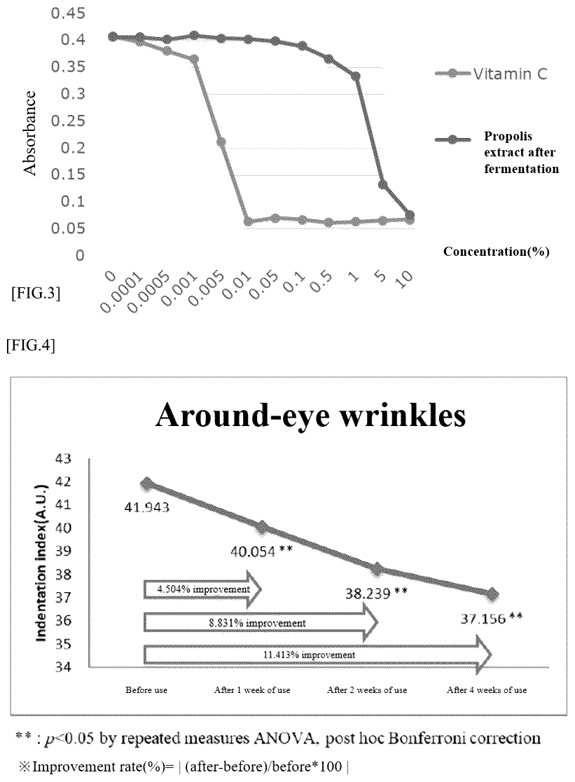

As a result of evaluation, as shown in FIG. 3, it can be seen that, although the activity of the fermented green propolis extract was lower than that of vitamin C (ascorbic acid) which is a representative powerful antioxidant substance, an antioxidant effect was exhibited.

### Experimental Example 4: Wrinkle Improvement Effect of Fermented Green Propolis Extract

Using an ampoule-type cosmetic containing 9 wt % of the fermentation green propolis as in Example 3, a total of 23 subjects were tested for the effect of the product on improvement in around-eye wrinkles, under-eye wrinkles, nasolabial folds, and neck wrinkles for a total of 4 weeks.

Use of Test Product

The test was made by performing measurement before use, after 1 week of use, after 2 weeks of use, and after 4 weeks of use, and the first test before use prohibited the use of basic products for 12 hours before the visit. Also, during the human application test period, the use of cosmetics containing an active ingredient that may affect test results (e.g., functional cosmetics such as whitening and wrinkle improvement cosmetics) other than the test product was prohibited. This test was based on use of the product twice a day in the morning and evening for 4 weeks.

Measurement and Analysis Methods of Around-Eye Wrinkles/Under-Eye Wrinkles/Nasolabial Folds/Neck Wrinkles

Measurement of around-eye wrinkles/under-eye wrinkles/nasolabial folds/neck wrinkles was made by photographing the same sites (area around the left eye, area under the left eye, left nasolabial area, and central neck area) of the subject before and after use of the test product using Antera 3D CS (Miravex Ltd., Ireland) which is a 3D skin photographing device. The saved image was converted to wrinkle medium mode, and analysis ranges including a circle with a diameter of 10.7 mm for the analysis of around-eye wrinkles, lines for the analysis of under-eye wrinkles and nasolabial folds, and a circle with a diameter of 24.0 mm for the analysis of neck wrinkles were designated. For around-eye wrinkles and neck wrinkles, an indentation index value representing the average roughness (Ra) of wrinkles in the range was analyzed and indicated in A.U. For under-eye wrinkles and nasolabial folds, a depth value representing the average depth of wrinkles in the range was analyzed and indicated in mm. As the indentation index (A.U.) and the depth (mm) values decrease, it means that there is an effect of improving around-eye wrinkles/under-eye wrinkles/nasolabial folds/neck wrinkles.

Test Results

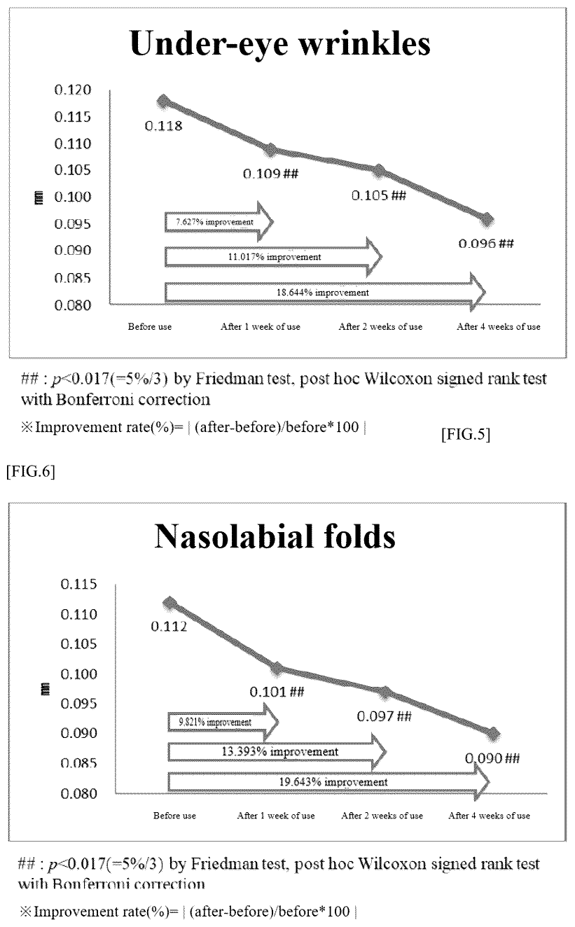

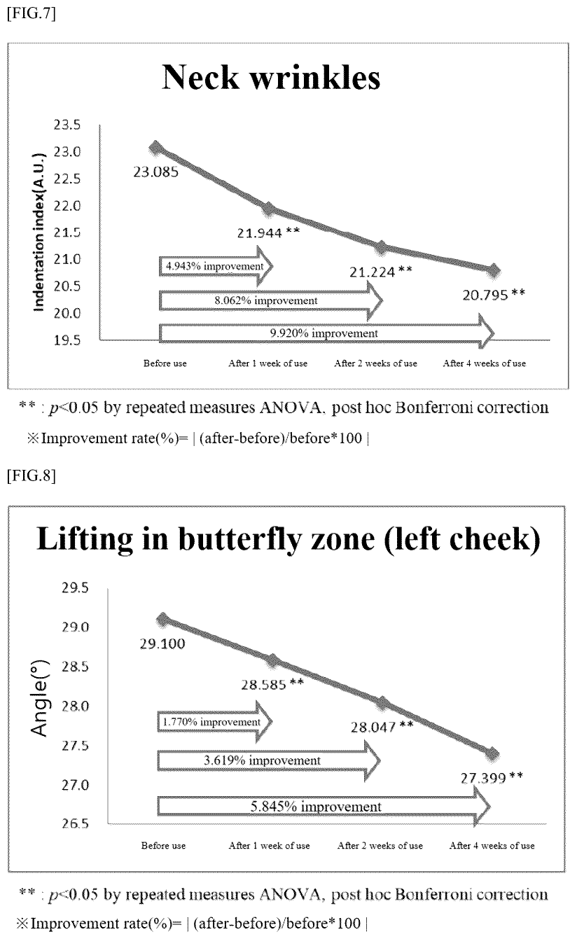

As shown in FIGS. 4 to 7, it was confirmed that the test sites exhibited wrinkle improvement rates for around-eye wrinkles: 4.504%, under-eye wrinkles: 7.627%, nasolabial folds: 9.821%, and neck wrinkles: 4.943% after 1 week of use; around-eye wrinkles: 8.831%, under-eye wrinkles: 11.017%, nasolabial folds: 13.393%, and neck wrinkles: 8.062% after 2 weeks of use; and around-eye wrinkles: 11.413%, under-eye wrinkles: 18.644%, nasolabial folds: 19.643%, and neck wrinkles: 9.920% after 4 weeks of use.

In conclusion, a statistically significant level of wrinkle improvement effect was shown after 1 week of use.

### Experimental Example 5: Lifting Improvement Effect of Fermented Green Propolis Extract

In order to evaluate effectiveness as a cosmetic material, using an ampoule-type cosmetic containing 9 wt % of the fermentation green propolis as in Example 3, a total of 23 subjects were tested for the effect of the product on lifting in the butterfly zone, around the mouth, around the eyes, in the chin area, in the nasolabial area, and in the double chin area for a total of 4 weeks.

Use of Test Product

The test was made by performing measurement before use, after 1 week of use, after 2 weeks of use, and after 4 weeks of use, and the first test before use prohibited the use of basic products for 12 hours before the visit. Also, during the human application test period, the use of cosmetics containing an active ingredient that may affect test results (e.g., functional cosmetics such as whitening and wrinkle improvement cosmetics) other than the test product was prohibited. This test was based on use of the product twice a day in the morning and evening for 4 weeks.

Measurement and Analysis Methods of Lifting in Butterfly Zone, Around Mouth, Around Eyes, and in Chin Area

Measurement of lifting in the butterfly zone, around the mouth, around the eyes, and in the chin area was made by photographing the same face of the subject before and after use of the test product using F-ray (BEYOUNG Co., Ltd., Korea) which is a device that expresses the appearance of skin as a contour line, and measuring a lifting level based on the curves formed near each test site. Lifting in the butterfly zone was measured by analyzing both cheeks in the image taken from the 30° side using Image-Pro Plus (Media Cybernetics, USA). The lifting was evaluated by drawing a first straight line downward from the center of the circle formed near the cheekbone; drawing a second straight line from the starting point at which the first straight line meets the bottom contour line to the inner boundary point at which it comes in contact with the pattern with the opposite color to the starting circle at the place where it moves 3 steps to the right along the horizontal stripes drawn on the contour picture; and then measuring the angle)(° between the two straight lines. As the angle)(° between the two straight lines decreases, it means that there is a lifting improvement effect in the butterfly zone.

Lifting in the chin area was measured by analyzing the image taken from the 45° side using Image-Pro Plus (Media Cybernetics, USA). The lifting was evaluated by drawing a first straight line from the area around the mouth to the chin, drawing a second straight line from the area around the eyes to the chin, connecting two points at the chin so that two straight lines are connected, and then measuring the angle at the chin. As the angle decreases, it means that there is an effect of lifting a sagging chin.

Measurement and Analysis Methods of Lifting in the Nasolabial Area

Measurement of lifting in the nasolabial area was made by photographing the same left nasolabial area of the subject before and after use of the test product using Antera 3D CS (Miravex Ltd., Ireland) which is a 3D skin photographing device. The saved image was converted to depression medium mode, the analysis range including the corresponding area was designated using a line, and the volume (mm 3) of a depressed area in the range was analyzed. As the volume (mm 3) of the depressed area decreases, it means that there is a lifting improvement effect in the nasolabial area.

Measurement and Analysis Methods of Lifting in the Double Chin Area

Measurement of lifting in the double chin area was made by photographing the same double chin area of the subject before and after use of the test product using Vectra XT (Canfield Imaging Systems, USA) which is able to measure skin volume through a 3D image, and analyzing the obtained image. The volume (ml) of the double chin area in the saved image was analyzed. As the volume (ml) decreases, it means that there is a lifting improvement effect in the double chin area.

Test Results

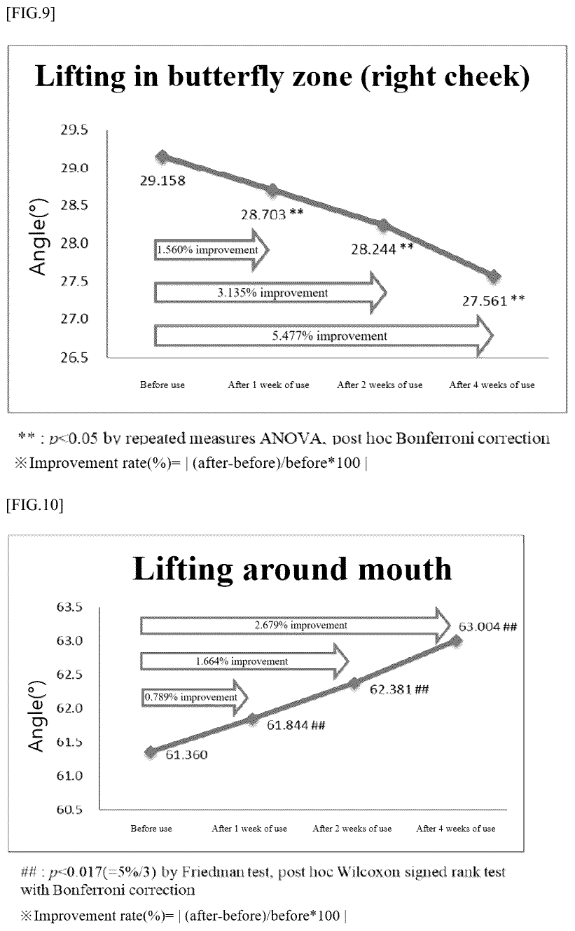

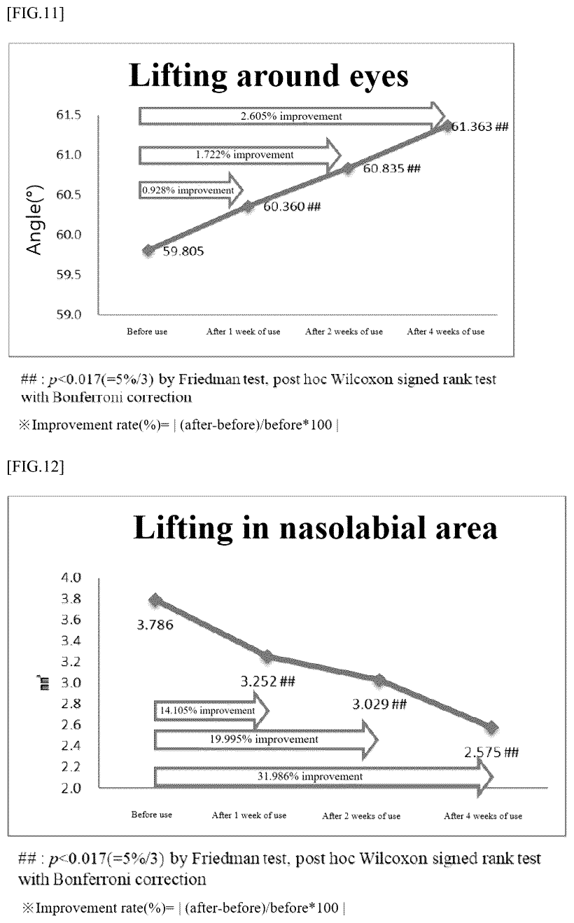

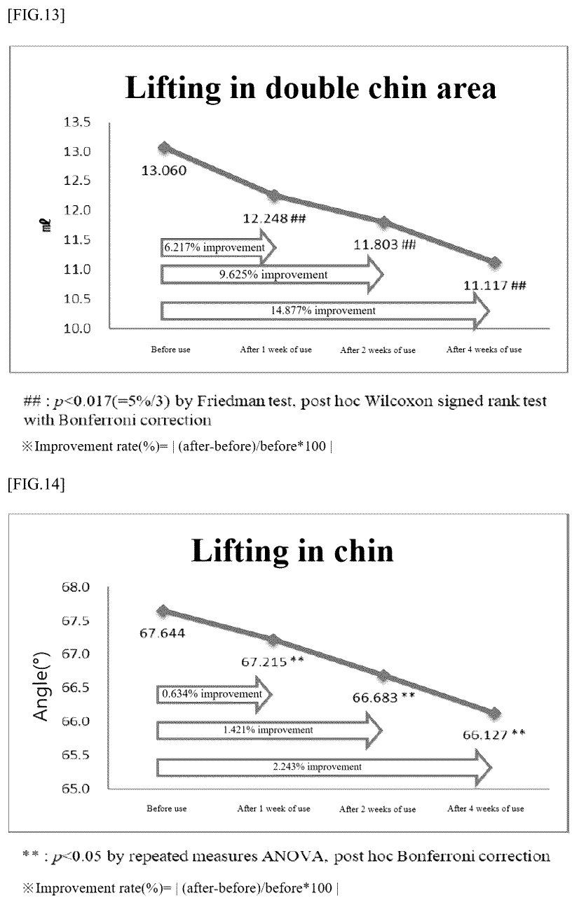

As shown in FIGS. 8 to 14, it was confirmed that the test sites exhibited lifting improvement rates for the butterfly zone (left): 1.770%, the butterfly zone (right): 1.560%, the area around the mouth: 0.789%, the area around the eyes: 0.928%, the nasolabial area: 14.105%, the double chin area: 6.217%, and the chin area: 0.634% after 1 week of use; the butterfly zone (left): 3.619%, the butterfly zone (right): 3.135%, the area around the mouth: 1.664%, the area around the eyes: 1.722%, the nasolabial area: 19.995%, the double chin area: 9.625%, and the chin area: 1.421% after 2 weeks of use; and the butterfly zone (left): 5.845%, the butterfly zone (right): 5.477%, the area around the mouth: 2.679%, the area around the eyes: 2.605%, the nasolabial area: 31.986%, the double chin area: 14.877%, and the chin area: 2.243% after 4 weeks of use.

In conclusion, a statistically significant level of lifting improvement effect was shown after 1 week of use.

### Experimental Example 6: Elasticity Improvement Effect of Fermented Green Propolis Extract

In order to evaluate effectiveness as a cosmetic material, using an ampoule-type cosmetic containing 9 wt % of the fermentation green propolis as in Example 3, a total of 23 subjects were tested for the effect of the product on improvement in skin density and elasticity for a total of 4 weeks.

Use of Test Product

The test was made by performing measurement before use, after 1 week of use, after 2 weeks of use, and after 4 weeks of use, and the first test before use prohibited the use of basic products for 12 hours before the visit. Also, during the human application test period, the use of cosmetics containing an active ingredient that may affect test results (e.g., functional cosmetics such as whitening and wrinkle improvement cosmetics) other than the test product was prohibited. This test was based on use of the product twice a day in the morning and evening for 4 weeks.

Measurement and Analysis Methods of Skin Density

Measurement of skin density was made by analyzing images obtained by measuring the same area around the left eye of the subjects before and after use of the test product using a DUB-USB skin scanner (TPM taberna pro-medicum, Germany). The measurement parameter of skin density is density, and the units thereof are %. As the measured value increases, it means that there is a skin density improvement effect.

Measurement and Analysis Methods of Skin Elasticity

Measurement of skin elasticity was made by measuring the same right cheek area of the subjects before and after use of the test product using Cutometer MPA580 (Courage+Khazaka electronic GmbH, Germany). The elasticity of the skin was measured, and a constant negative pressure was applied to the inside of a probe vertically attached to the skin to measure the degrees of skin tension and restoration. The measurement was performed three times, and the average value thereof was used as skin elasticity evaluation data. As an analysis parameter R2 value, which is gross elasticity, is closer to 1, it means that the skin is more elastic.

Test Results

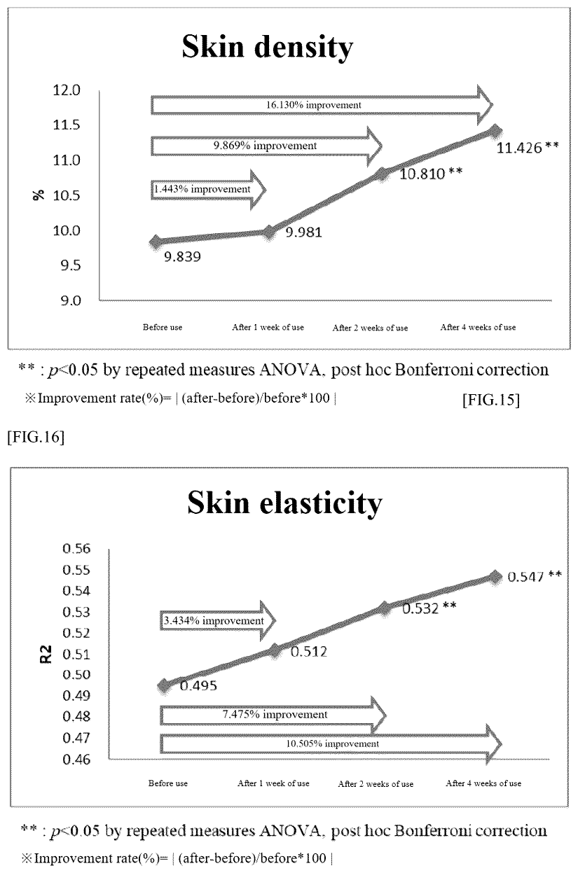

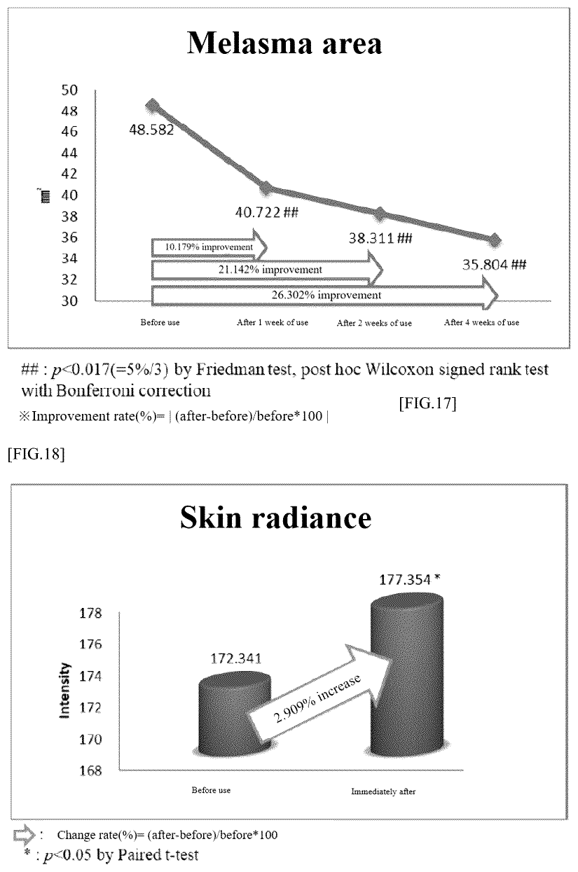

As shown in FIGS. 15 and 16, it was confirmed that the test sites exhibited skin elasticity improvement rates for skin density: 1.443% and elasticity: 3.434% after 1 week of use; skin density: 9.869% and elasticity: 7.475% after 2 weeks of use; and skin density: 16.139% and elasticity: 10.505% after 4 weeks of use.

In conclusion, a statistically significant level of elasticity improvement effect was shown after 2 weeks of use.

### Experimental Example 7: Melasma Improvement and Skin Radiance Improvement Effects of Fermented Green Propolis Extract

In order to evaluate effectiveness as a cosmetic material, using an ampoule-type cosmetic containing 9 wt % of the fermentation green propolis as in Example 3, a total of 23 subjects were tested for the effect of the product on melasma improvement for a total of 4 weeks, and a test for a skin radiance effect was conducted with one-time use of the product.

Use of Test Product

The test was made by performing measurement before use, after 1 week of use, after 2 weeks of use, and after 4 weeks of use, and the first test before use prohibited the use of basic products for 12 hours before the visit. Also, during the human application test period, the use of cosmetics containing an active ingredient that may affect test results (e.g., functional cosmetics such as whitening and wrinkle improvement cosmetics) other than the test product was prohibited. This test was based on use of the product twice a day in the morning and evening for 4 weeks. In the case of radiance evaluation, skin radiance was measured after one-time use.

Measurement and Analysis Methods of Skin Melasma Area/Skin Radiance

Measurement of melasma area was made by photographing the same skin melasma site of the left cheek of the subject before and after use of the test product using Antera 3D CS (Miravex Ltd., Ireland) which is a 3D skin photographing device. The resulting image was converted to melanin mode, the analysis range (d=44.8 mm) was designated, and a melasma area (mm²) in the range was analyzed. As the area value decreases, it means that there is a melasma area improvement effect.

Measurement of skin radiance was made by photographing the same face of the subject before and after use of the test product using VISIA-CR (Canfield Imaging Systems, USA). The parallel-mode image was analyzed using Image-Pro Plus (Media Cybernetics, USA), a specific area in the cheek was designated, and then an intensity value was analyzed. As the intensity value increases, it means that there is a skin radiance improvement effect.

Test Results

As shown in FIGS. 17 and 18, it was confirmed that the test sites exhibited a melasma area improvement rate of 16.179% after 1 week of use, 21.14% after 2 weeks of use, and 26.302% after 4 weeks of use, and skin radiance was improved 2.909% after one-time use.

In conclusion, a statistically significant level of melasma improvement effect was shown after 1 week of use, and the skin radiance improvement effect was shown immediately after use.

### Experimental Example 8: Moisturizing Improvement Effect of Fermented Green Propolis Extract

In order to evaluate effectiveness as a cosmetic material, using an ampoule-type cosmetic containing 9 wt % of the fermentation green propolis as in Example 3, a total of 23 subjects were tested for the effect of the product on skin moisturizing for a total of 4 weeks.

Use of Test Product

The test was made by performing measurement before use, after 1 week of use, after 2 weeks of use, and after 4 weeks of use, and the first test before use prohibited the use of basic products for 12 hours before the visit. Also, during the human application test period, the use of cosmetics containing an active ingredient that may affect test results (e.g., functional cosmetics such as whitening and wrinkle improvement cosmetics) other than the test product was prohibited. This test was based on use of the product twice a day in the morning and evening for 4 weeks.

Measurement and Analysis Methods of Skin Moisturizing

Measurement of skin moisturizing was made by measuring the same right cheek area of the subjects before and after use of the test product using Corneometer CM825 (Courage+Khazaka electronic GmbH, Germany). During the measurement, a Corneometer probe was brought in contact with the skin, measurement was performed three time through a sensor, and the average value thereof was used as skin moisturizing evaluation data. The Corneometer is a device that basically considers skin as an insulator and utilizes the property of conducting electricity better when more moisture is contained, and measures capacitance at the point where the probe touches. Therefore, a moisture content and capacitance are proportional to each other, and a higher measurement value means a higher moisture content. The unit is an arbitrary unit (A.U.) which is a unit constant.

Measurement and Analysis Methods of Deep Moisturizing

Measurement of deep moisturizing was made by measuring the same right cheek area of the subjects before and after use of the test product using XS5 (0.5 mm) of probes of the MoistureMeter D (Delfin Technologies Ltd, Finland). An evaluation value is measured using EM wave, the unit is an arbitrary unit (A.U.) which is a unit constant, and a higher measurement value means that there is a deep moisturizing improvement effect.

Test Results

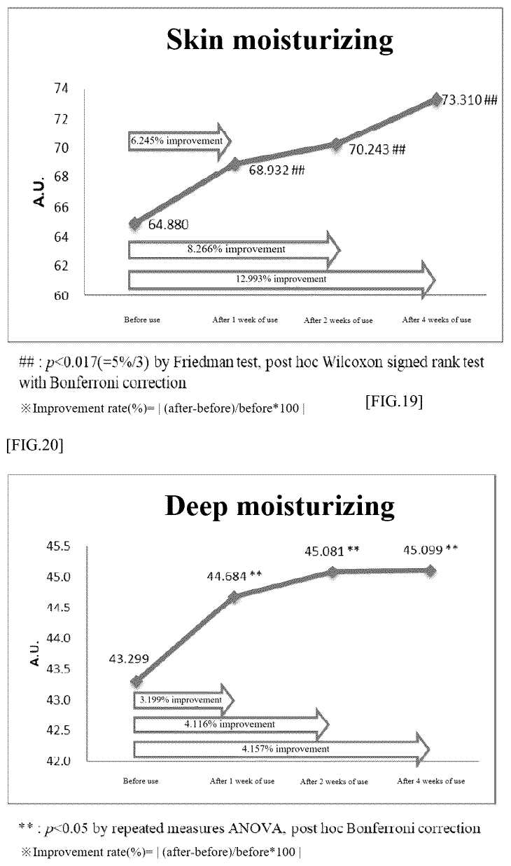

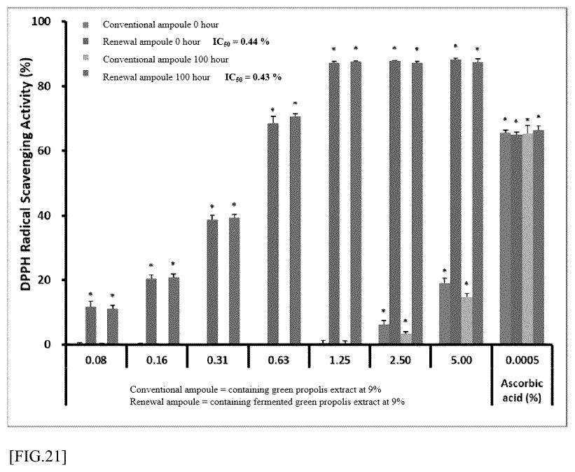

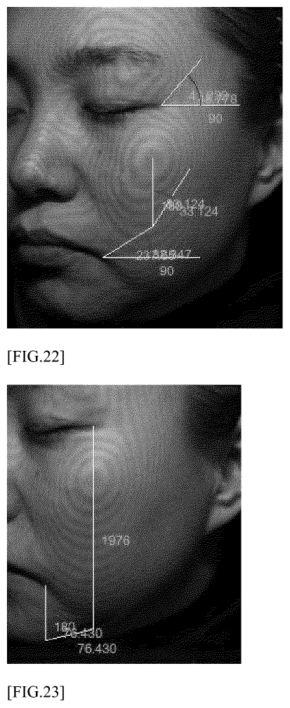

As shown in FIGS. 19 and 20, it was confirmed that the test sites exhibited skin moisturizing improvement rates of moisturizing: 6.245% and deep moisturizing: 3.199% after 1 week of use; moisturizing: 8.266% and deep moisturizing: 4.116% after 2 weeks of use; and moisturizing: 12.993% and deep moisturizing: 4.157% after 4 weeks of use.

In conclusion, a statistically significant level of skin moisturizing improvement effect was shown after 1 week of use.

### Experimental Example 9: Antioxidant Enhancement Effect of Fermented Green Propolis Extract

It was confirmed from the above-described result of Experimental Example 3 and FIG. 3 that the fermented green propolis extract of the present invention exhibited an antioxidant effect. Meanwhile, the antioxidant activities of the fermented green propolis extract (Example 3) of the present invention and a general green propolis extract (Comparative Example 2) were compared.

Material

DPPH used in this experiment was purchased from Sigma-Aldrich Inc.

Sample Processing Method

A green propolis ampoule and a fermentation green propolis ampoule were allowed to stand at high temperature for 0 hour and 100 hours, and then diluted with methanol so that a final concentration became 0.08, 0.16, 0.31, 0.63, 1.25, 2.50, and 5.00% according to each time point.

Measurement of Antioxidant Activity (DPPH Radical Scavenging Activity)

The antioxidant activity of each sample extract was measured by a DPPH free radical scavenging method according to a Blois method (Blois, 1958). Each sample was diluted with MeOH to prepare various concentrations, 500 μl of each diluted sample was dispensed into a microtube, an equal amount of 300 uM DPPH dissolved in methanol was added, and then the microtube was allowed to stand at 37° C. for 30 minutes. 200 μl of the supernatant was dispensed into a 96-well plate, and absorbance at 517 nm was measured using an ELISA reader. As a positive control, L-ascorbic acid (Asc.A) was prepared at 0.0005%, and absorbance was measured and compared with that of the sample. A lower absorbance of the reaction mixture indicates higher free radical scavenging activity. DPPH free radical scavenging activity was calculated by the following equation, and antioxidant activity was expressed as the reducing power by electron donating ability (EDA %). This experiment was repeated three times for each sample, and the average value thereof was obtained.

DPPH free radical scavenging activity (%)={(Control absorbance−Sample absorbance)/Control absorbance}×100

Sample absorbance=Absorbance of the reaction solution having the sample added thereto

Control absorbance=Absorbance of the reaction solution having methanol added thereto instead of the sample

Statistical Analysis

Data was expressed as mean value±standard deviation, the experimental results were the results of three or more independent experiments, and statistical significance was verified at the significance level of p<0.05 by an independent t-test.

Results

As an experimental result, it was confirmed that the antioxidant effect of an ampoule containing a fermented green propolis extract at 9% was superior to that of an ampoule containing a general green propolis extract at 9%, and the conventional ampoule (containing a green propolis extract) exhibited reduced antioxidant activity when allowed to stand at high temperature for 100 hours, whereas the ampoule containing a fermentation green propolis at 9% exhibited IC₅₀ values of 0.44% at 0 hour and 0.43% at 100 hours, and thus antioxidant activity was maintained well even at high temperature.

1.-18. (canceled)

19. A method of preparing a fermented propolis extract, the method comprising:
a first step of subjecting propolis wax to cold extraction at 5° C. to 40° C. to prepare a propolis extract;
a second step of subjecting the propolis extract extracted in the first step to ultrasonic extraction; and
a third step of fermenting the extract obtained in the second step with a fermentation strain.

20. The method according to claim 19, further comprising a fourth step of performing solvent extraction with 1,3-butylene glycol in an amount of 20 to 40 wt % based on the total weight of the extract fermented in the third step.

21. The method according to claim 19, wherein the fermented propolis extract has an increased artepillin C content.

22. The method according to claim 19, wherein the cold extraction is solvent extraction with a C1 to C5 alcohol.

23. The method according to claim 19, wherein the ultrasonic extraction in the second step is performed at a wavelength of 10 kHz to 40 kHz and a temperature of 10° C. to 40° C.

24. The method according to claim 19, wherein the propolis is one or more selected from the group consisting of green propolis, red propolis, and brown propolis.

25. The method according to claim 19, wherein the fermentation strain in the third step is one or more selected from the group consisting of Lactobacillus acidophilus, Lactobacillus casei, Lactobacillus bulgaricus, Lactobacillus rhamnosus, Lactobacillus plantarum, Lactobacillus reuteri, Lactobacillus confusus, Lactobacillus fermentum, Lactobacillus brevis, and Thermus thermophilus.

26. The method according to claim 19, wherein the fermentation in the third step is culturing for 65 to 80 hours.

27. A fermented propolis extract prepared by the method according to claim 19.

28. A composition comprising the fermented propolis extract prepared by the method according to claim 19 as an active ingredient.

29. The composition according to claim 28, wherein the composition is used for improving skin, wherein the improvement of skin is selected from the group consisting of improvement of skin wrinkles or skin elasticity, skin whitening, skin antioxidation and anti-inflammation.

30. The composition according to claim 28, wherein the composition is selected from cosmetics, foods, and quasi-drugs.

31. A method of improving skin, comprising:
administering or applying a composition including a fermented propolis extract to a subject or subject's skin and improving skin in the subject or subject's skin, wherein the improvement of skin is selected from the group consisting of improvement of skin wrinkles or skin elasticity, skin whitening, skin antioxidation and anti-inflammation.

32. The method according to claim 31, wherein the fermented propolis extract has an increased artepillin C content.

33. The method according to claim 31, wherein the propolis is one or more selected from the group consisting of green propolis, red propolis, and brown propolis.

34. The method according to claim 31, wherein the fermented propolis extract is included in an amount of 0.01 to 15 wt % relative to the total weight of the cosmetic composition.

35. The method according to claim 31, wherein the fermentation is performed with one or more strains selected from the group consisting of Lactobacillus acidophilus, Lactobacillus casei, Lactobacillus bulgaricus, Lactobacillus rhamnosus, Lactobacillus plantarum, Lactobacillus reuteri, Lactobacillus confusus, Lactobacillus fermentum, Lactobacillus brevis, and Thermus thermophilus.
